(19) United States
(12) Patent Application Publication
(10) Pub. No.: US 2026/0334920 A1
(43) Pub. Date: Oct. 29, 2026

(54) NAVIGATING AN IMAGING INSTRUMENT TO CAPTURE AN AREA OF INTEREST

(71) Applicant: Biosense Webster (Israel) Ltd., Yokneam (IL)

(72) Inventors: Nadav Barnea, Haifa (IL); Hanna Cohen-Sacomsky, Zichron Ya'acov (IL); Vitaly Gutkovich, Haifa (IL); Morris Ziv-Ari, Atlit (IL)

(21) Appl. No.: 19/193,739

(22) Filed: Apr. 29, 2025

(51) Int. Cl. A61B 8/00; A61B 8/08; A61B 8/12

(52) U.S. Cl. CPC A61B 8/5207; A61B 8/0883; A61B 8/12; A61B 8/42; A61B 8/5292

(57) ABSTRACT

In one general aspect, methods may include receiving a ultrasound data captured via an imaging instrument having an instrument position and an instrument orientation. Methods may include determining, based at least on the ultrasound data and an imaging model, that the ultrasound data is not sufficient to generate a three dimensional (3D) model having a threshold amount of an area of interest for an anatomical structure. Methods may include causing output, based at least on the ultrasound data and the imaging model, of information indicative of one or more of a change in the instrument position or the instrument orientation configured to result in a capture of another ultrasound data sufficient to generate a 3D model having the threshold amount.

### BACKGROUND

An imaging instrument, such as an intravascular ultrasound catheter, may be used to capture images of a body part, such as the heart. However, insertion and movement of the imaging instrument may cause complications, such as dissection, perforation, arrhythmia, thrombosis, and vasospasm.

Improvements are needed.

### SUMMARY

It is to be understood that both the following general description and the following detailed description are exemplary and explanatory only and are not restrictive. Methods and systems for navigating an imaging device to capture images of an area of interest (e.g., region of an anatomical structure) are described.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, methods may include receiving a plurality of first three dimensional (3D) models. Each of the first 3D models may include a threshold amount of an area of interest of an anatomical structure such as the left atrium of a heart, for example. Other areas or structure of interest may be used. Methods may include generating, based at least on a portion of the plurality of first 3D models, a plurality of first ultrasound data. Each of the first ultrasound data may include a first instrument position and a first instrument orientation. Methods may include training, based at least on the plurality of first ultrasound data, an imaging model. Methods may include receiving a second ultrasound data captured via an imaging instrument having a second instrument position and a second instrument orientation. Methods may include determining, based at least on the second ultrasound data and the imaging model, that the second ultrasound data does not satisfy a threshold requirement to generate a second 3D model having the threshold amount. Methods may include causing output, based at least on the second ultrasound data and the imaging model, of information indicative of one or more of a change in the second instrument position or the second instrument orientation configured to result in a capture of a third ultrasound data having the threshold amount. Other embodiments of this aspect include corresponding computer systems, apparatus, and computer programs recorded on one or more computer storage devices, each configured to perform the actions of the methods.

In one general aspect, methods may include receiving a plurality of first three dimensional (3D) models. Each of the first 3D models may include a threshold amount of an area of interest of an anatomical structure such as the left atrium of a heart, for example. Methods may include generating, based at least on a portion of the plurality of first 3D models, a plurality of first ultrasound data. Each of the first ultrasound data may include a first instrument position and a first instrument orientation. Methods may include training, based at least on the plurality of first ultrasound data, an imaging model. Methods may include receiving a second ultrasound data captured via an imaging instrument having a second instrument position and a second instrument orientation. Methods may include determining, based at least on the second ultrasound data and the imaging model, that the second ultrasound data satisfies a threshold requirement to generate a second 3D model having the threshold amount. Methods may include generating, based on the second ultrasound data, a second 3D model having the threshold amount. Other embodiments of this aspect include corresponding computer systems, apparatus, and computer programs recorded on one or more computer storage devices, each configured to perform the actions of the methods.

In one general aspect, methods may include receiving a ultrasound data captured via an imaging instrument having an instrument position and an instrument orientation. Methods may include determining, based at least on the ultrasound data and an imaging model, that the ultrasound data is not sufficient to generate a three dimensional (3D) model having a threshold amount of an area of interest of an anatomical structure such as the left atrium of a heart, for example. Methods may include causing output, based at least on the ultrasound data and the imaging model, of information indicative of one or more of a change in the instrument position or the instrument orientation configured to result in a capture of another ultrasound data sufficient to generate a 3D model having the threshold amount. Other embodiments of this aspect include corresponding computer systems, apparatus, and computer programs recorded on one or more computer storage devices, each configured to perform the actions of the methods.

In one general aspect, systems may include one or more processors configured to: receive a plurality of first three dimensional (3D) models, where each of the first 3D models may include a threshold amount of an area of interest of an anatomical structure such as the left atrium of a heart; generate, based at least on a portion of the plurality of first 3D models, a plurality of first ultrasound data, where each of the first ultrasound data may include a first instrument position and a first instrument orientation; train, based at least on the plurality of first ultrasound data, an imaging model; receive a second ultrasound data captured via an imaging instrument having a second instrument position and a second instrument orientation; determine, based at least on the second ultrasound data and the imaging model, that the second ultrasound data does not satisfy a threshold requirement to generate a second 3D model having the threshold amount; and cause output, based at least on the second ultrasound data and the imaging model, of information indicative of one or more of a change in the second instrument position or the second instrument orientation configured to result in a capture of a third ultrasound data having the threshold amount. Other embodiments of this aspect include corresponding computer systems, apparatus, and computer programs recorded on one or more computer storage devices, each configured to perform the actions of the methods.

In one general aspect, systems may include one or more processors configured to: receive a plurality of first three dimensional (3D) models, where each of the first 3D models may include a threshold amount of a left atrium of a heart; generate, based at least on a portion of the plurality of first 3D models, a plurality of first ultrasound data, where each of the first ultrasound data may include a first instrument position and a first instrument orientation; train, based at least on the plurality of first ultrasound data, an imaging model; receive a second ultrasound data captured via an imaging instrument having a second instrument position and a second instrument orientation; determine, based at least on the second ultrasound data and the imaging model, that the second ultrasound data satisfies a threshold requirement to generate a second 3D model having the threshold amount; and generate, based on the second ultrasound data, a second 3D model having the threshold amount. Other embodiments of this aspect include corresponding computer systems, apparatus, and computer programs recorded on one or more computer storage devices, each configured to perform the actions of the methods.

In one general aspect, systems may include one or more processors configured to: receive a ultrasound data captured via an imaging instrument having an instrument position and an instrument orientation; determine, based at least on the ultrasound data and an imaging model, that the ultrasound data is not sufficient to generate a three dimensional (3D) model having a threshold amount of an area of interest of an anatomical structure such as the left atrium of a heart; and cause output, based at least on the ultrasound data and the imaging model, of information indicative of one or more of a change in the instrument position or the instrument orientation configured to result in a capture of another ultrasound data sufficient to generate a 3D model having the threshold amount. Other embodiments of this aspect include corresponding computer systems, apparatus, and computer programs recorded on one or more computer storage devices, each configured to perform the actions of the methods.

These and other features and advantages are described in greater detail below.

### BRIEF DESCRIPTION OF THE DRAWINGS

Some features are shown by way of example, and not by limitation, in the accompanying drawings. In the drawings, like numerals reference similar elements.

FIG. 1 shows an example user interface described herein.

FIG. 2 shows an example user interface described herein.

FIG. 3 shows an example user interface described herein.

FIG. 4 shows a flowchart of an example method disclosed herein.

FIG. 5 shows a flowchart of an example method disclosed herein.

FIG. 6 shows a flowchart of an example method disclosed herein.

FIGS. 7A-7D show stages of creating simulated ultrasound contours from an example 3D model.

FIGS. 8A-8D show voxelization and sampling of an example 3D model.

FIGS. 9A-9E show detail obtained from various sampling ratios of an example 3D model.

FIG. 10 shows a layout for an encoder and/or decoder disclosed herein.

FIG. 11 shows an example network architecture for a convolutional neural network (CNN) disclosed herein.

The accompanying drawings show examples of the disclosure. It is to be understood that the examples shown in the drawings and/or discussed herein are non-exclusive and that there are other examples of how the disclosure may be practiced.

### DETAILED DESCRIPTION

The accompanying drawings, which form a part hereof, show examples of the disclosure. It is to be understood that the examples shown in the drawings and/or discussed herein are non-exclusive and that there are other examples of how the disclosure may be practiced.

The present disclosure relates to navigating an imaging device to capture images of an area of interest of an anatomical structure such as a left atrium of a heart, for example. Other areas or structures may be used. Reference is made throughout this disclosure to the left atrium of the heart as a non-limiting, illustrative example. Although conventional problems with imaging such structures as the left atrium of the heart may be addressed by the present disclosure, such references are not intended to limit the applicability of the systems and methods of the present disclosure to other structure or areas of anatomical structures. The description, figures, and functionality described herein, as applied to areas of the heart, may be applied to other areas and structures without departing from the spirit of the invention.

In certain cardiac imaging and reconstruction a 3-dimensional cardiac reconstruction is carried out by catheterizing a heart using a probe with a mapping electrode, and acquiring electrical data from respective locations in areas of interest in the heart, representing the locations of the electrical data as a point cloud, reconstructing a model of the heart from the point cloud, applying a set of filters to the model to produce a filtered volume, segmenting the filtered volume to define components of the heart, and reporting the segmented filtered volume, such as described in U.S. Pat. No. 9,265,434B2, which is hereby incorporated herein by reference in its entirety for any and all purposes. As a non-limiting example, such electroanatomical mapping (e.g., Fast Anatomical Mapping (FAM)) may be accomplished by the CARTO® 3 System cooperatively with a mapping catheter such as the Navistar® Thermocool® catheter, both available from Biosense Webster, Inc., 3333 Diamond Canyon Road, Diamond Bar, Calif. 91765. Other systems and catheters (e.g., mapping catheters) may be used in this manner.

As noted, segmentation may be required to separate a volume into the structure(s) of interest (SOI). In certain cases, the SOI is the area of most interest for real-time mapping, such as the left atrium or left ventricle (or other structures). In the context of cardiac imaging, segmentation of the left atrium may be necessary for various purposes, including volumetric analysis, functional assessment, and treatment planning, for example.

However, in certain applications imaging tools (e.g., ultrasound (ULS) catheters or systems) may be used where no or limited electroanatomical mapping is available. The present disclosure relates to systems and methods to generate an anatomical model (e.g., shell, 3D model) without the need of a diagnostic/mapping catheter, but instead using only the ultrasound data from an ultrasound catheter.

As a non-limiting example, one or more 3D models may be identified to include the entire left atrium (or at least a threshold amount of an area or structure of interest). From the one or more 3D models, ultrasound data may be generated to represent (e.g., as raw ultrasound data or visualization data) a region of interest, such as the left atrium, from different angles. A model, such as a machine learning or probabilistic model may be developed (e.g., trained, tested, tuned) using the ultrasound data as a representation of what target ultrasound data should represent when the entire area or structure of interest (e.g., left atrium) is successfully captured. As such, the model may then be implemented to provide feedback to users of whether inputted ultrasound data are satisfactory or not to capture a threshold portion of the left atrium.

As an example, in a real-time procedure, a user may capture ultrasound data of the left atrium using a catheter such as a ULS catheter. Using the subject model, one may predict if the entire left atrium would be represented in the imaging field. If yes, then a 3D model may be generated including a threshold amount of the left atrium. If no, then instruction may be provided to instruct the user how to adjust the catheter in order to be in better position for viewing the threshold amount of the left atrium. Other structures of interest may be used.

As a further illustrative example, a plurality of three dimensional (3D) models may be received. The plurality of 3D models may each comprise a 3D model of a threshold amount of the area of interest of an anatomical structure such as the left atrium of a heart. The threshold amount may be 100%. Planar images may be generated from the plurality of 3D models. Ultrasound data may be generated from the plurality of 3D models. A ultrasound data may comprise two orthogonal planes. A generated ultrasound data may represent a ultrasound data an imaging instrument may have taken of an associated area of interest (e.g., left atrium) of an associated heart had the imaging instrument been used with an associated instrument position and an associated instrument orientation. The generated ultrasound data, associated instrument positions, and associated instrument orientations may be used to train an imaging model. The imaging model may take in a ultrasound data as input. The imaging model may use statistical probability to infer an instrument position and/or instrument orientation based on the input. The imaging model may use statistical probability infer if the imaging instrument comprises an instrument position and/or instrument orientation to capture sufficient imaging information to create a 3D model of an area of interest such as an associated left atrium of an associated heart.

The systems and/or methods disclosed herein may use feature extraction for model accuracy. Feature extraction may comprise using planar cross-sections, surface texture analysis, edge detection, etc. The systems and/or methods disclosed herein may use corrective feedback mechanisms for model completion. Corrective feedback mechanisms may comprise positional adjustments, orientation guidance, real-time alerts, etc. Using feature extraction and/or corrective feedback may improve an accuracy of left atrium imaging, reduce left atrium imaging procedure time, enhance overall clinical outcomes, etc. Other structures and areas of interest of the body may have associated features for extractions.

The systems and/or methods disclosed herein may use planar cross-sections for model accuracy. For example, the systems and/or methods disclosed herein may extract orthogonal planar cross-sections of the structure or area of interest to analyze coverage and completeness of the structure or area of interest within an imaging field. The extracted orthogonal planar cross-sections of the structure or area of interest may be analyzed to identify anatomical landmarks. For example, when the structure or area of interest is the left atrium of a heart, the anatomical landmarks may comprise pulmonary veins, a left atrial appendage, etc. Other anatomical landmarks may be used for the left atrium or other structures or areas of interest. Identification of the anatomical landmarks may confirm that an entire structure or area of interest is within the imaging field.

The systems and/or methods disclosed herein may use surface texture analysis for model accuracy. For example, a model may be examined for texture and contour of a surface of a structure or area of interest, such as a left atrium surface. A comparison may be performed between the examined texture and contour of the surface of the structure or area of interest and known templates of complete and/or incomplete models of structures or areas of interest. Based on the comparison, the systems and/or methods described herein may identify missing sections of the structure or area of interest and/or sections of the structure or area of interest that were poorly captured by the model. The systems and/or methods disclosed herein may prompt corrective action based on the missing sections and/or sections that were poorly captured.

The systems and/or methods disclosed herein may use edge detection algorithms to highlight boundaries of a structure or area of interest, such as a left atrium, in a model. Highlighting the boundaries of the structure or area of interest in the model helps a user ensure that the structure or area of interest is sufficiently captured, which helps ensure that the model is complete. Missing and/or unclear edges in the model may trigger feedback for the user to adjust a position and/or orientation of a catheter.

The systems and/or methods disclosed herein may use positional adjustments for model completion. The systems and/or methods described herein may extract features from a model, as explained above. If the extracted features indicate an incomplete capture of a structure or area of interest, the systems and/or methods may provide specific feedback on how to reposition an imaging instrument. For example, the systems and/or methods disclosed herein may provide feedback indicating a particular rotation (e.g., clockwise, slightly clockwise, clockwise 15 degrees) or lateral movement (e.g., forward, slightly forward, forward 2 millimeters) to ensure imaging of all areas of interest of the structure or area, such as a left atrium.

The systems and/or methods disclosed herein may use orientation guidance for model completion. The systems and/or methods described herein may guide a user in adjusting a tilt or orientation of an imaging instrument to better align with a structure of a left atrium. Structures of the left atrium that may be of value include a mitral valve, pulmonary veins, etc. The systems and/or methods described herein may provide guidance (e.g., deflect to the left, deflect slightly to the left, deflect 5 degrees to the left) to obtain clear imaging of the structures of the left atrium. Other structures or areas of interest may be used.

The systems and/or methods disclosed herein may use real-time alerts for model completion. During an imaging process, the systems and/or methods described herein may detect whether a structure or area of interest, such as a left atrium, is captured sufficiently. If the systems and/or methods described herein detect that the structure or area of interest is captured insufficiently, then the systems and/or methods described herein may generate real-time alerts to prompt a user to make corrections. Using real-time alerts may eliminate a need for repeat scan and enhance an accuracy of a final 3D model.

The systems and/or methods described herein may determine that a left atrium appendage is not fully visible in a current ultrasound data. Other structures or areas of interest may be used. The systems and/or methods described herein may create feedback advising adjustment an imaging instrument by rotating the imaging instrument 20 degrees counter-clockwise.

The systems and/or methods described herein may determine that pulmonary veins are only partially captured. Other structures or areas of interest may be used. The systems and/or methods described herein may create feedback suggesting retraction of an imaging instrument slightly and increasing a tilt angle of the imaging instrument by 7 degrees to improve visibility.

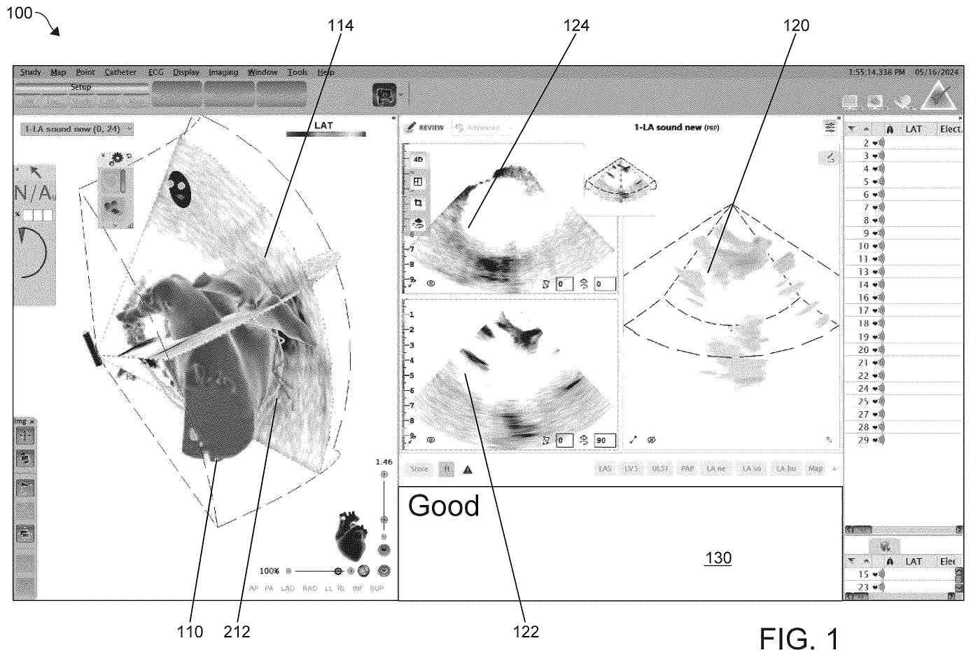

FIG. 1 shows an example user interface 100 described herein. The user interface may be associated with an imaging system such as the CARTO® 3 System by BIOSENSE WEBSTER, INC. Other systems and user interfaces may be used. One or more of a 3D model 110, a first planar image 112, a second planar image 114, a first image 122, a second image 124, or an imagining field 120 may be presented via the user interface 100. As an example, the user interface 100 may present one or more of the 3D model 110, the first planar image 112, the second planar image 114, the first image 122, the second image 124, or the imagining field 120 over time. The outputs of the user interface 100 may update in or near real-time. Although ultrasound data in FIG. 1 is shown with reference to four dimensional (4D) volumetric images and planar images, other ultrasound data is contemplated, such as ultrasound data representing or converted to one or more two dimensional (2D) images, etc. Additionally, the ultrasound data may or may not be represented on a display. For example, the ultrasound data may be or comprise raw ultrasound beam data.

The 3D model may be an anatomical model or shell that represents an imaged portion of an anatomical structure. As shown, the 3D model 110 comprises an overlayed first planar image 112 and second planar image 114. The first planar image 112 may be orthogonal to the second planar image 114. The first planar image 112 and the second planar image 114 may be created by an imaging instrument such as an intravascular ultrasound catheter. The imaging instrument may be configured to capture image data from inside a structure or area of interest, such as a heart, such as inside or adjacent a left atrium of the heart. The first planar image 112 may be based on a position and/or orientation of the imaging instrument. The second planar image 114 may be based on the position and/or orientation of the imaging instrument. The first planar image 112 and/or the second planar image 114 may be used to create the 3D model 110.

As described herein, the first planar image 112 and/or the second planar image 114 may be representative of ultrasound data that may be generated from the data of the 3D model 110 to visualize an area or structure of interest (e.g., the left atrium) from different angles. As further described herein, the 3D model 110 may represent one of a plurality of 3D models from which the ultrasound data may be generated to visualize the structure or area of interest from different angles. A model, such as a machine learning or probabilistic model may be developed (e.g., trained, tested, tuned) using the ultrasound data (e.g., the first planar image 112 and/or the second planar image 114) as a representation of what target ultrasound data should look like when the entire structure or area of interest is successfully captured. As an example, the model (e.g., neural network) may be configured to use feature extraction from multi-plane slices of the 3D models (e.g. Biplane or Biplane slices from 4D). As such, the model may then be implemented to provide feedback to users of whether inputted ultrasound data are satisfactory or not to capture a threshold portion of the structure or area of interest.

Various 3D models such as the example 3D model 110 may be stored. Information included in the 3D models or captured as reference data to the 3D models may be indicative of various ultrasound data taken at various positions and orientations. Ultrasound data may comprise multi-planar images such as the first planar image 112 and/or the second planar image 114 may be stored. Information included in the ultrasound information may include one or more of position or orientation of the image. Such example 3D models and multi-planar images may be used as the knowledge base (e.g., training or testing set) to develop a model described herein. Other sources of imaging data for developing the model may be used.

While the first planar image 112 and/or the second planar image 114 may be represented overlaying the 3D model 110, information represented by the first planar image 112 may be displayed in first image 122 and information represented by the second planar image 114 may be displayed in second image 124.

Presented another way, a user may capture ultrasound data of the structure or area of interest using a catheter such as a ULS catheter. As an example, a user interface such as the user interface 100 may be configured to output a representation of the imaging data captured by the catheter. As a further example, first image 122 and the second image 124 may represent ULS data captured by the catheter from various orientations or positions. The data captured to generate one or more of the first image 122 and the second image 124 may be used to generate an imaging field 120, which may be configured to present a 4D representation of the ULS data showing 3D imagery data over time. Other images may be used.

Using the subject model, one may predict if the threshold amount of the structure or area of interest would be represented in the imaging field 120 as the user captures the ultrasound data. As an illustrative example, the imaging field 120 may represent a 3D volume of an area or structure over time (e.g., 4D volume) and the % coverage of the structure or area of interest may be determined from the volume represented in the imaging field 120. If the threshold amount of the structure or area of interest is represented in the 3D volume in the imaging field 120, then a 3D model may be generated including a threshold amount of the structure or area of interest. If the threshold amount of the structure or area of interest is not represented in the 3D volume in the imaging filed 120, then instruction may be provided to instruct the user how to adjust the catheter in order to be in better position for viewing the threshold amount of the structure or area of interest.

For example, the user interface 100 may comprise a feedback message 130. The feedback message 130 may comprise a message regarding corrective positioning and/or corrective orientation of the imaging device. For example, the feedback message 130 may indicate that the imaging device should be deflected away from a septum. The 3D model 110 may comprise at least a portion of the structure or area of interest. The feedback message 130 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied. The message regarding corrective positioning and/or corrective orientation of the imaging device may comprise instructions intended to put the imaging instrument in a position to take a multi-planar image that may create a 3D model that satisfies the threshold amount.

As shown, for example, in FIG. 1, the feedback message 130 may comprise a message confirming a satisfactory position and/or satisfactory orientation of the imaging device. For example, the feedback message 130 may indicate that the imaging device comprises a good position and/or good orientation. The 3D model 110 may comprise at least a portion of the structure or area of interest. The feedback message 130 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied.

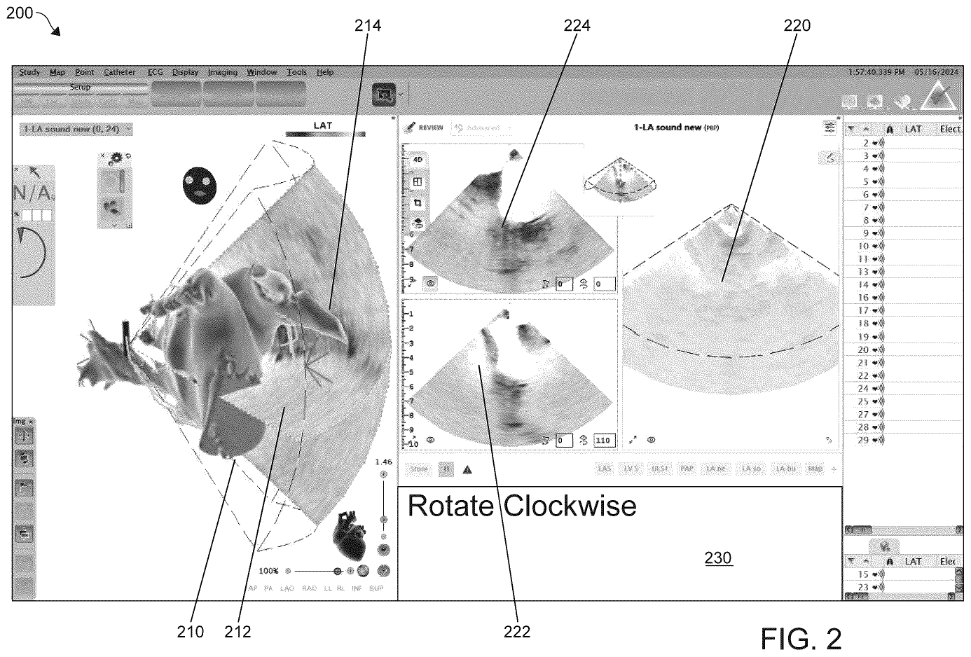

FIG. 2 shows an example user interface 200 described herein. The user interface 200 may similar elements to the user interface 100 in FIG. 1. The user interface 200 may comprise a 3D model 210 comprising a first planar image 212 and a second planar image 214, a first image 222 corresponding to the first planar image 212, a second image 224 corresponding to the second planar image 214, a visual display 220 of the 3D model 210 changing over time, and a feedback message 230. The feedback message 230 may a comprise message regarding corrective positioning and/or corrective orientation of the imaging device. For example, the feedback message 230 may indicate that the imaging device should be rotated clockwise. The 3D model 210 may comprise at least a portion of the structure or area of interest, such as the left atrium of the heart. The feedback message 230 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied. The message regarding corrective positioning and/or corrective orientation of the imaging device may comprise instructions intended to put the imaging instrument in a position to take a multi-planar image that may create a 3D model that satisfies the threshold amount.

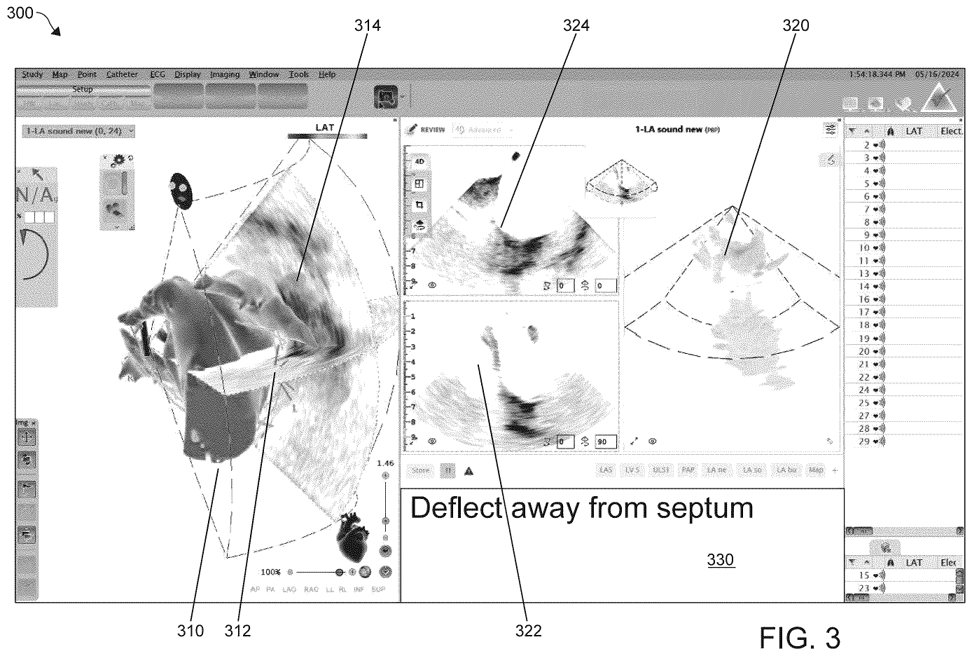

FIG. 3 shows an example user interface 300 described herein. The user interface 300 may similar elements to the user interface 100 in FIG. 1 and the user device 200 in FIG. 2. The user interface 300 may comprise a 3D model 310 comprising a first planar image 312 and a second planar image 314, a first image 322 corresponding to the first planar image 312, a second image 324 corresponding to the second planar image 314, a visual display 320 of the 3D model 310 changing over time, and a feedback message 330. The user interface 300 may show elements after instructions regarding the imagining instructions in the feedback message 230 in FIG. 2 are followed. The feedback message 330 may a comprise message regarding corrective positioning and/or corrective orientation of the imaging device. For example, the feedback message 330 may indicate that the imaging device should be deflected away from the septum. The 3D model 310 may comprise at least a portion of the structure or area of interest, such as the left atrium of the heart. The feedback message 330 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied. The message regarding corrective positioning and/or corrective orientation of the imaging device may comprise instructions intended to put the imaging instrument in a position to take a multi-planar image that may create a 3D model that satisfies the threshold amount.

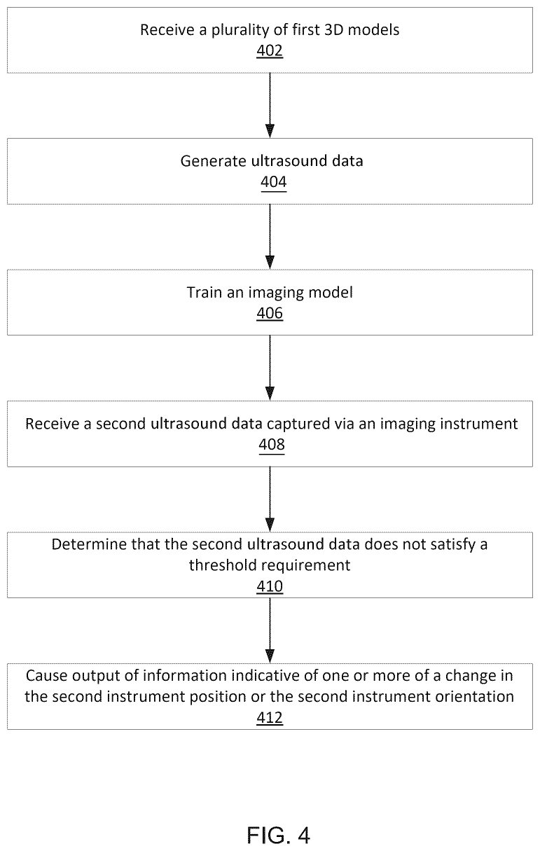

FIG. 4 is a flowchart of an example process 400. In some implementations, one or more process blocks of FIG. 4 may be performed by a computing device. The computing device may create the images shown in FIGS. 1-3. The computing device may be in communication with a second computing device, wherein the second computing device may create the images shown in FIGS. 1-3.

As shown in FIG. 4, process 400 may include receiving a plurality of first three dimensional (3D) models (block 402). For example, a computing device may receive a plurality of first 3D models. Each of the first 3D models may include a threshold amount of a structure or area of interest, such as a left atrium of a heart. The threshold amount may comprise 100%. As a further example, the first 3D models may comprise a training set or testing set of data relating to the development of a probabilistic model or learning algorithm. The first 3D models may comprise ULS 3D data and/or anatomical mapping (e.g., FAM, CT, or the like).

As shown in FIG. 4, process 400 may include generating a plurality of first ultrasound data (block 404). For example, the computing device may generate a plurality of first ultrasound data. The plurality of first ultrasound data may be generated based at least on a portion of the plurality of first 3D models. Each of the first ultrasound data may include a first instrument position and a first instrument orientation. The ultrasound data may be or comprise raw ultrasound beam data. The ultrasound data may or may not be represented on a display. The ultrasound data may represent or be converted to one or more of four dimensional (4D) volumetric images, one or more two dimensional (2D) images, one or more multi-plane images, etc.

As shown in FIG. 4, process 400 may include training an imaging model (block 406). For example, the computing device may train an imaging model. The imaging model may be trained based at least on the plurality of first ultrasound data.

As shown in FIG. 4, process 400 may include receiving a second ultrasound data captured via an imaging instrument (block 408). For example, the computing device may receive a second ultrasound data captured via an imaging instrument. The imaging instrument may have a second instrument position and a second instrument orientation. The receiving a second ultrasound data captured via an imaging instrument may comprise receiving the second ultrasound data in or near real-time as the second ultrasound data is captured via the imaging instrument. The imaging instrument may be configured to emit ultrasound waves.

As shown in FIG. 4, process 400 may include determining that the second ultrasound data does not satisfy a threshold requirement (block 410). For example, the computing device may determine that the second ultrasound data does not satisfy a threshold requirement. Determining that the second ultrasound data does not satisfy a threshold requirement may be based at least on the second ultrasound data and the imaging model. The threshold requirement may be and/or comprise a threshold requirement to generate a second 3D model having the threshold amount. The determining that the second ultrasound data does not satisfy a threshold requirement may comprise determining that the second ultrasound data does not have sufficient planar information for the imaging model to generate a full 3D model.

As shown in FIG. 4, process 400 may include causing output of information indicative of one or more of a change in the second instrument position or the second instrument orientation (block 412). For example, the computing device may cause output of information indicative of one or more of a change in the second instrument position or the second instrument orientation. The output may be caused based at least on the second ultrasound data and the imaging model. The information indicative of one or more of a change in the second instrument position or the second instrument orientation may be configured to result in a capture of a third ultrasound data having the threshold amount.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

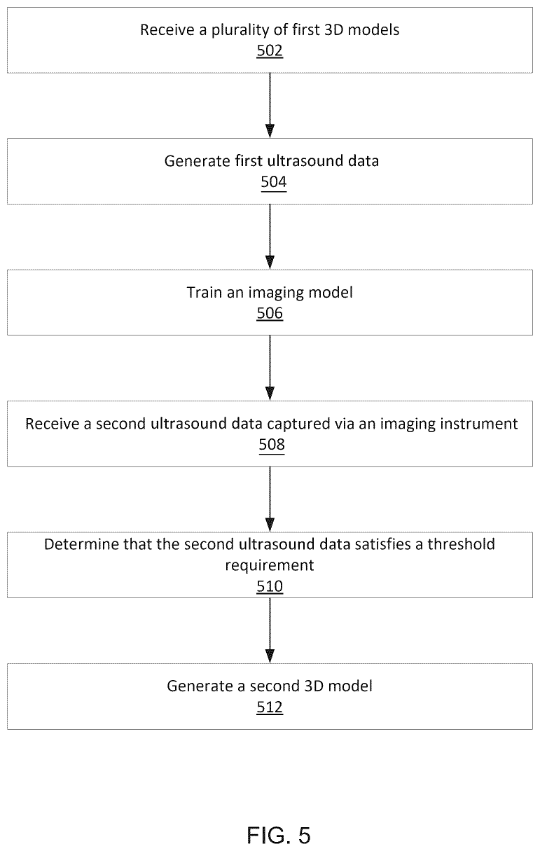

FIG. 5 is a flowchart of an example process 500. In some implementations, one or more process blocks of FIG. 5 may be performed by a computing device. The computing device may create the images shown in FIGS. 1-3. The computing device may be in communication with a second computing device, wherein the second computing device may create the images shown in FIGS. 1-3.

As shown in FIG. 5, process 500 may include receiving a plurality of first three dimensional (3D) models (block 502). For example, a computing device may receive a plurality of first 3D models. Each of the first 3D models may include a threshold amount of a structure or area of interest, such as a left atrium of a heart. The threshold amount may comprise 100%.

As shown in FIG. 5, process 500 may include generating a plurality of first ultrasound data (block 504). For example, the computing device may generate a plurality of first ultrasound data. The plurality of first ultrasound data may be generated based at least on a portion of the plurality of first 3D models. Each of the first ultrasound data may include a first instrument position and a first instrument orientation. The ultrasound data may be or comprise raw ultrasound beam data. The ultrasound data may or may not be represented on a display. The ultrasound data may represent or be converted to one or more of four dimensional (4D) volumetric images, one or more two dimensional (2D) images, one or more multi-plane images, etc.

As shown in FIG. 5, process 500 may include training an imaging model (block 506). For example, the computing device may train an imaging model. The imaging model may be trained based at least on the plurality of first ultrasound data.

As shown in FIG. 5, process 500 may include receiving a second ultrasound data captured via an imaging instrument (508). For example, the computing device may receive a second ultrasound data captured via an imaging instrument. The imaging instrument may have a second instrument position and a second instrument orientation. The receiving a second ultrasound data captured via an imaging instrument may comprise receiving the second ultrasound data in or near real-time as the second ultrasound data is captured via the imaging instrument. The imaging instrument may be configured to emit ultrasound waves.

As shown in FIG. 5, process 500 may include determining that the second ultrasound data satisfies a threshold requirement (block 510). The computing device may determine that the second ultrasound data satisfies a threshold requirement. Determining that the second ultrasound data satisfies a threshold requirement may be based at least on the second ultrasound data and the imaging model. The threshold requirement may be and/or comprise a threshold requirement to generate a second 3D model having the threshold amount. The determining that the second ultrasound data satisfies a threshold requirement may comprise determining that the second ultrasound data has sufficient planar information for the imaging model to generate a full 3D model.

As shown in FIG. 5, process 500 may include generating a second 3D model (block 512). For example, the computing device may generate a second 3D model. The 3D model may be generated based on the second ultrasound data.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

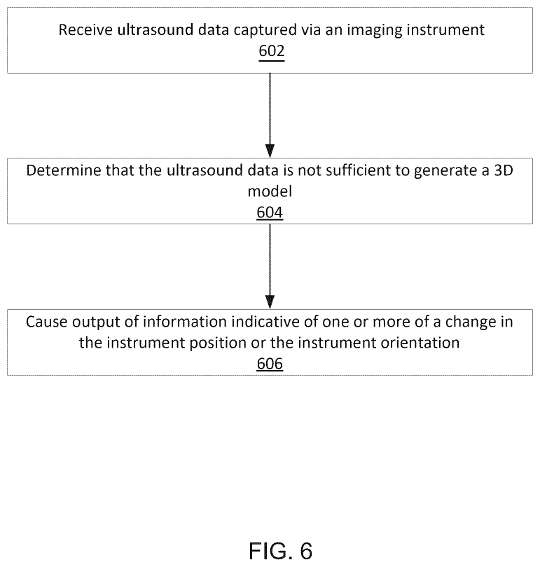

FIG. 6 is a flowchart of an example process 600. In some implementations, one or more process blocks of FIG. 6 may be performed by a computing device. The computing device may create the images shown in FIGS. 1-3. The computing device may be in communication with a second computing device, wherein the second computing device may create the images shown in FIGS. 1-3.

As shown in FIG. 6, process 600 may include receiving a ultrasound data captured via an imaging instrument (block 602). For example, a computing device may receive a ultrasound data captured via an imaging instrument. The imaging instrument may have an instrument position and an instrument orientation. The imaging instrument may be configured to emit ultrasound waves. The ultrasound data may be or comprise raw ultrasound beam data. The ultrasound data may or may not be represented on a display. The ultrasound data may represent or be converted to one or more of four dimensional (4D) volumetric images, one or more two dimensional (2D) images, one or more multi-plane images, etc.

As shown in FIG. 6, process 600 may include determining that the ultrasound data is not sufficient to generate a three dimensional (3D) model (block 604). For example, the computing device may determine that the ultrasound data is not sufficient to generate a 3D model. The determining that the ultrasound data is not sufficient to generate a 3D model may be based at least on the ultrasound data and an imaging model. The determining that the ultrasound data is not sufficient to generate a 3D model may comprise determining that the ultrasound data is not sufficient to generate a 3D model having a threshold amount of a structure or area of interest, such as a left atrium of a heart. The imaging model may be trained using a plurality of first ultrasound data. The plurality of first ultrasound data may comprise an associated instrument position and an associated instrument orientation. The plurality of first ultrasound data may have been generated based on a plurality of 3D models. The plurality of 3D models may comprise an associated threshold amount of an associated structure or area of interest. The threshold amount may be 100%.

As shown in FIG. 6, process 600 may include causing output of information indicative of one or more of a change in the instrument position or the instrument orientation (block 606). For example, the computing device may cause output of information indicative of one or more of a change in the instrument position or the instrument orientation. The output may be caused based at least on the ultrasound data and the imaging model. The information indicative of one or more of a change in the instrument position or the instrument orientation may be configured to result in a capture of another ultrasound data sufficient to generate a 3D model having the threshold amount.

Process 600 may comprise generating a new 3D model comprising the threshold amount of the structure or area of interest. For example, the computing device may generate a new 3D model comprising the threshold amount of the structure or area of interest. Process 600 may comprise producing at least one new ultrasound data based on the new 3D model. For example, the computing device may produce at least one new ultrasound data based on the new 3D model. Process 600 may comprise determining, based on the at least one new ultrasound data, if the determining that the ultrasound data is not sufficient to generate a 3D model comprising a threshold amount of the structure or area of interest was correct. For example, the computing device may determine, based on the at least one new ultrasound data, if the determining that the ultrasound data is not sufficient to generate a 3D model comprising a threshold amount of the structure or area of interest was correct.

Process 600 may include determining, based on the at least one new ultrasound data, that the determining that the ultrasound data is not sufficient to generate a 3D model comprising a threshold amount of the structure or area of interest was incorrect. For example, the computing device may determine, based on the at least one new ultrasound data, that the determining that the ultrasound data is not sufficient to generate a 3D model comprising a threshold amount of the structure or area of interest was incorrect. Process 600 may include updating the imaging model based on the new 3D model and the at least one new ultrasound data. For example, the computing device may update the imaging model based on the new 3D model and the at least one new ultrasound data.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

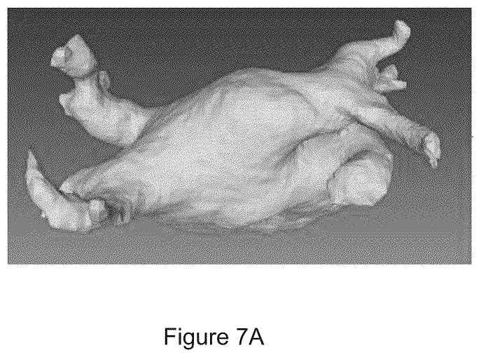

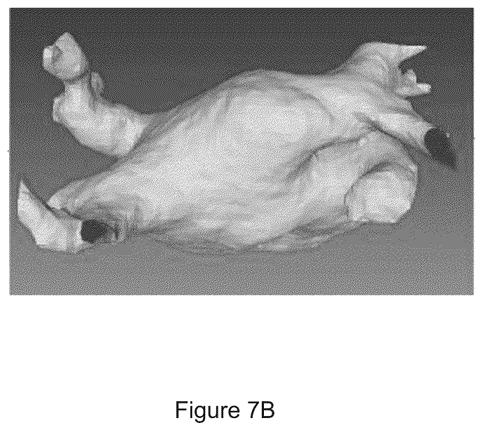

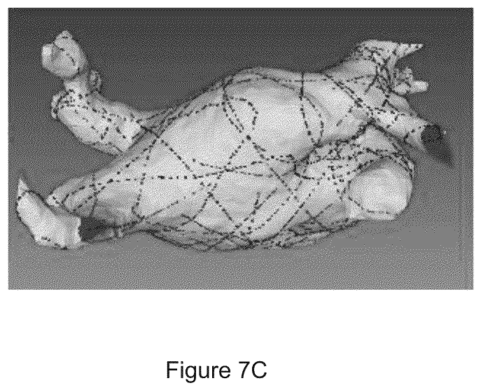

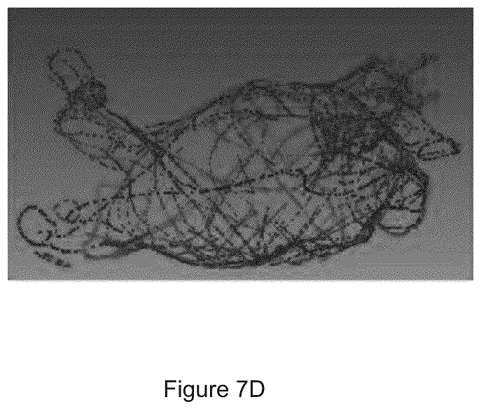

The systems and/or methods described herein may automatically generate an anatomical model of a structure or area of interest, such as a heart, or a portion of a heart, such as a chamber, such as a left atrium of a heart, based on ultrasound contours. A model creation algorithm may be created by using deep-learning training to associate simulated ultrasound contours with three-dimensional (3D) models. 3D models may comprise 3D images from computer topography (CT) scans. The stages of creating simulated ultrasound contours may be illustrated in FIGS. 7A-7D. FIG. 7A shows an existing 3D model of a heart, which may have been acquired via known methods. FIG. 7B shows the model of FIG. 7A after the model has been trimmed. The model may be trimmed by a predetermined amount in each axis. The predetermined amount may be 5%. Trimming the model may remove a pulmonary veins region not sampled in an ultrasound contour.

FIG. 7C shows a number of two-dimensional (2D) planes (slices, etc.) through the 3D model. The 2D planes may be generated at random angles. The 2D planes may simulate contours acquired by an ultrasound instrument. The number of 2D planes shown in FIG. 7C is 35, although other numbers of 2D planes may be used, as explained below. FIG. 7D shows a final sampling. The final sampling may be a result of sampling a percentage of a surface of the 3D model. The percentage may be 80%. The final sampling of each plane may simulate a plane field of view for an ultrasound instrument. FIG. 7D may simulate 35 ultrasound contours taken at random angles. FIG. 7D may be given as example input for the deep-learning training, and FIG. 7A and/or FIG. 7B may be given as example associated expected output for the example input.

The systems and/or methods described herein may perform voxelization of the contours to generate a 3D binary matrix. The voxelization may be performed because deep-learning may perform better with images and matrices as opposed to 3D meshes. A voxelization resolution may be set. For example, the voxelization resolution may be set to 0.5 millimeters. The voxelization resolution may be set based on computational resource considerations, such as graphics processing unit (GPU) memory limitations.

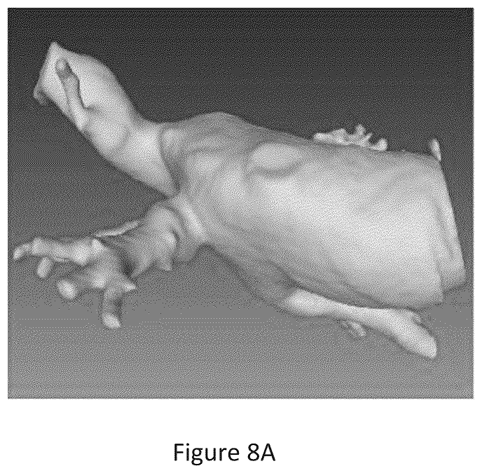

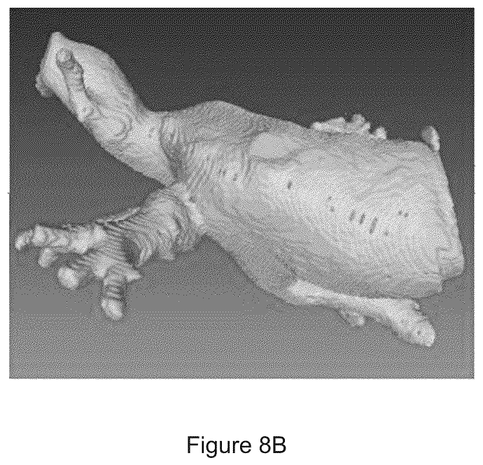

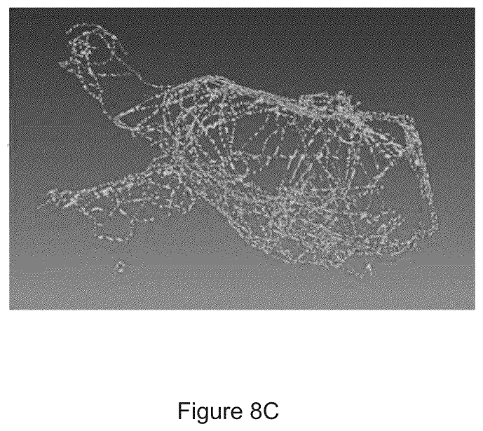

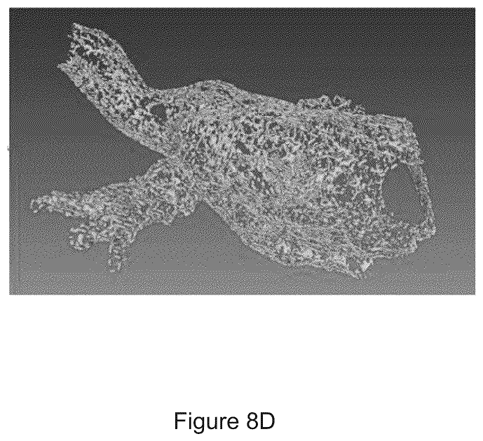

FIG. 8A shows an example 3D model from a CT scan. FIG. 8B shows an example voxelized model based on the 3D model in FIG. 8A. FIG. 8C shows a result of an example sampling of the voxelized model in FIG. 8B. The example sampling of FIG. 8C may represent 10% sampling ratio. The sampling ratio may be computed as a number of voxels used to represent the samplings in FIG. 8C divided by a number of voxels used to represent the voxelized model in FIG. 8B. FIG. 8D shows a result of an example sampling of the voxelized model in FIG. 8B. The example sampling of FIG. 8D may represent 30% sampling ratio. The sampling ratio may be computed as a number of voxels used to represent the samplings in FIG. 8D divided by a number of voxels used to represent the voxelized model in FIG. 8B. references to voxel and pixel are for illustration and may be used to reference 2D or 3D, as applicable, and without departing from the spirit of the present disclosure.

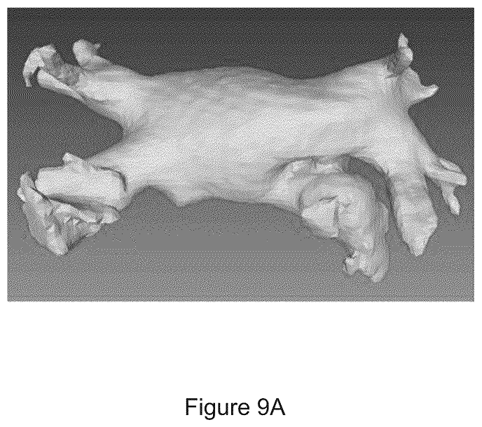

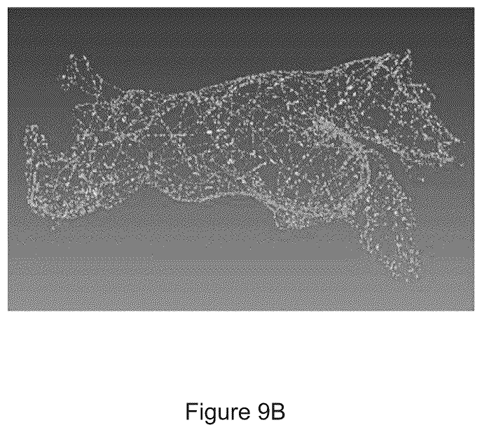

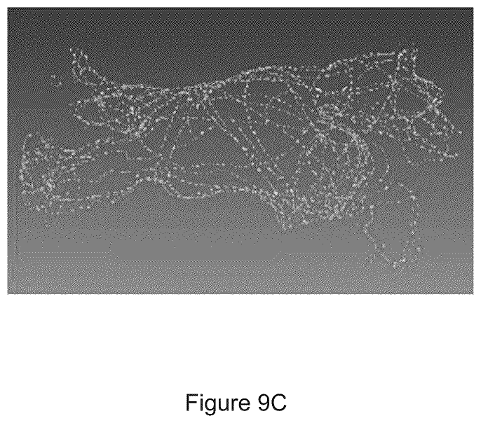

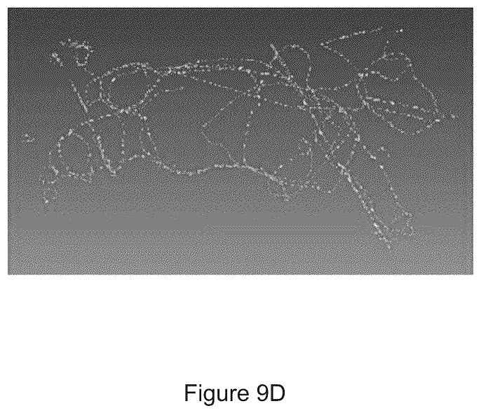

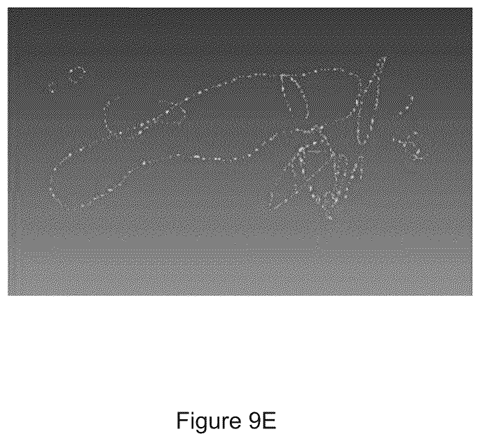

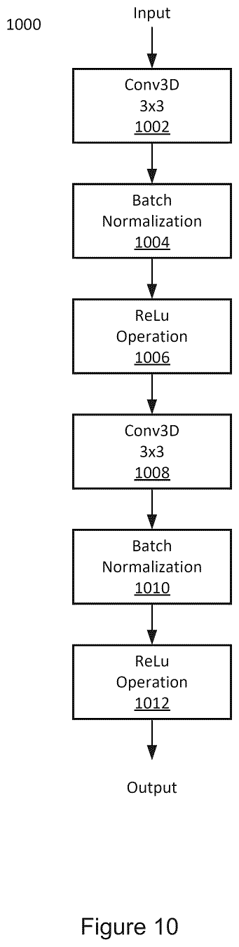

FIG. 9A shows an example 3D model from a CT scan. FIGS. 9B-9E demonstrate detail obtained from various sampling ratios of the 3D model in FIG. 9A. FIG. 9B shows 10% sampling, or about 50 planes (slices, etc.). FIG. 9C shows 5% sampling, or about 25 planes. FIG. 9D shows 3% sampling, or about 15 planes. FIG. 9E shows 1% sampling, or about 5 planes. Sampling ratios that replicate actual ultrasound instrument contours obtained in practice are about 3% to 5%. FIG. 10 shows a layout for an encoder and/or decoder 1000 disclosed herein. Each block shown in FIG. 10 may represent a logical block, such as a module, algorithm, etc. The encoder and/or decoder 1000 may comprise a first 3D convolutional logic block 1002. The first 3D convolutional logic block 1002 may comprise a 3×3 kernel. The first 3D convolutional logic block 1002 may transpose an input matrix into a first intermediate matrix. The input matrix may represent a two-dimensional (2D) image. The input matrix may represent a three-dimensional (3D) image. The input matrix may comprise dimensions of 160×128×128. The first intermediate matrix may represent a two-dimensional (2D) image. The first intermediate matrix may represent a three-dimensional (3D) image. The first intermediate matrix may comprise dimensions of 160×128×128. Other dimensions may be used.

After the first 3D convolutional logic block 1002, the first intermediate matrix may be provided to a first batch normalization logic block 1004. The first batch normalization logic block 1004 may use the first intermediate matrix to adjust normalization parameters for the first intermediate matrix and/or future input. The first batch normalization logic block 1004 may cause the first intermediate matrix to become a first normalized matrix.

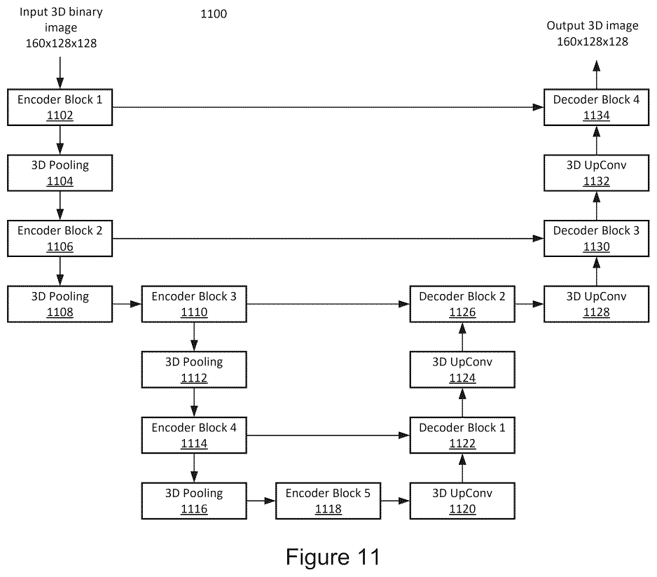

After the first batch normalization logic block 1004, the first normalized matrix may be provided to a first rectified linear unit (ReLu) operation block 1006. The first ReLu operation block 1006 may use the first normalized matrix to reenforce the encoder and/or decoder 1000 and/or an associated neural network, such as the neural network described in FIG. 11. The first ReLu operation block 1006 may cause the first normalized matrix to become a first updated matrix.

The first updated matrix may be provided as input to a second 3D convolutional logic block 1008. The second 3D convolutional logic block 1008 may comprise a 3×3 kernel. The second 3D convolutional logic block 1008 may transpose the first updated matrix into a second intermediate matrix. The first updated matrix may represent a two-dimensional (2D) image. The first updated matrix may represent a three-dimensional (3D) image. The first updated matrix may comprise dimensions of 160×128×128. The second intermediate matrix may represent a two-dimensional (2D) image. The second intermediate matrix may represent a three-dimensional (3D) image. The second intermediate matrix may comprise dimensions of 160×128×128. Other dimensions may be used.

After the second 3D convolutional logic block 1008, the second intermediate matrix may be provided to a second batch normalization logic block 1010. The second batch normalization logic block 1004 may use the second intermediate matrix to adjust normalization parameters for second first intermediate matrix and/or future input. The second batch normalization logic block 1010 may cause the second intermediate matrix to become a second normalized matrix.

After the second batch normalization logic block 1010, the second normalized matrix may be provided to a second ReLu operation block 1012. The second ReLu operation block 1012 may use the second normalized matrix to reenforce the encoder and/or decoder 1000 and/or an associated neural network, such as the neural network described in FIG. 11. The second ReLu operation block 1012 may cause the second normalized matrix to become a second updated matrix. The second updated matrix may be an output matrix. The output matrix may represent a two-dimensional (2D) image. The output matrix may represent a three-dimensional (3D) image. The output matrix may comprise dimensions of 160×128×128. Other dimensions may be used.

FIG. 11 shows an example network architecture for a convolutional neural network (CNN) 1100 disclosed herein. The CNN 1100 may comprise a plurality of encoders and decoders, like the encoder and/or decoder 1000 described in FIG. 10. The CNN 1100 may comprise 4 or 5 encoders and 4 decoders. The CNN 1100 may comprise a U shape. The CNN 1100 may comprise a plurality of 3D pooling logic blocks and a plurality of up-conversion logic blocks. The plurality of 3D pooling logic blocks and/or the plurality of up-conversion logic blocks may comprise a predetermined factor. For example, the predetermined factor may be 2. The CNN 1100 may be used to reconstruct 3D models.

The CNN 1100 may receive an image as input. The image may comprise a three-dimensional (3D) image. The image may comprise a binary image. The image may comprise a 160×128×128 matrix of voxels. A first encoder block 1102 may receive the image as input. The first encoder block 1102 may process the image to create a first processed image. The first encoder block 1102 may provide the first processed image to a first 3D pooling logic block 1104. The first encoder block 1102 may also provide the first processed image to a fourth decoder block 1134. The first 3D pooling logic block 1104 may process the first processed image to create a second processed image.

A second encoder block 1106 may receive the second processed image. The second encoder block 1106 may process the second processed image to create a third processed image. The second encoder block 1106 may provide the third processed image to a second 3D pooling logic block 1108. The second encoder block 1106 may also provide the third processed image to a third decoder block 1130. The second 3D pooling logic block 1108 may process the third processed image to create a fourth processed image.

A third encoder block 1110 may receive the fourth processed image. The third encoder block 1110 may process the fourth processed image to create a fifth processed image. The third encoder block 1110 may provide the fifth processed image to a third 3D pooling logic block 1112. The third encoder block 1110 may also provide the fifth processed image to a second decoder block 1126. The third 3D pooling logic block 1112 may process the fifth processed image to create a sixth processed image.

A fourth encoder block 1114 may receive the sixth processed image. The fourth encoder block 1114 may process the sixth processed image to create a seventh processed image. The fourth encoder block 1114 may provide the seventh processed image to a fourth 3D pooling logic block 1116. The fourth encoder block 1114 may also provide the seventh processed image to a first decoder block 1122. The fourth 3D pooling logic block 1116 may process the seventh processed image to create an eighth processed image.

A fifth encoder block 1118 may receive the eighth processed image. The fifth encoder block 1118 may process the sixth processed image to create a ninth processed image. The fifth encoder block 1118 may provide the ninth processed image to a first 3D up-conversion logic block 1120. The first 3D up-conversion logic block 1120 may process the ninth processed image to create a tenth processed image. The first 3D up-conversion logic block 1120 may provide the tenth processed image to the first decoder block 1122.

The first decoder block 1122 may process the seventh processed image and the tenth processed image to create an eleventh processed image. The first decoder block 1122 may provide the eleventh processed image to a second 3D up-conversion logic block 1124. The second 3D up-conversion logic block 1124 may process the eleventh processed image to create a twelfth processed image. The second 3D up-conversion logic block 1124 may provide the twelfth processed image to the second decoder block 1126.

The second decoder block 1126 may process the fifth processed image and the twelfth processed image to create a thirteenth processed image. The second decoder block 1126 may provide the thirteenth processed image to a third 3D up-conversion logic block 1128. The third 3D up-conversion logic block 1128 may process the thirteenth processed image to create a fourteenth processed image. The third 3D up-conversion logic block 1128 may provide the fourteenth processed image to the third decoder block 1130.

The third decoder block 1130 may process the third processed image and the fourteenth processed image to create a fifteenth processed image. The third decoder block 1130 may provide the fifteenth processed image to a fourth 3D up-conversion logic block 1132. The fourth 3D up-conversion logic block 1132 may process the fifteenth processed image to create a sixteenth processed image. The fourth 3D up-conversion logic block 1132 may provide the sixteenth processed image to the fourth decoder block 1134.

The fourth decoder block 1134 may process the first processed image and the sixteenth processed image to create a seventeenth processed image. The seventeenth processed image may be returned by the CNN 1100 as output. The seventeenth processed image may comprise a 3D image. The image may comprise a 160×128×128 matrix of voxels. Other dimension of matrices may be used.

The CNN 1100 may be implemented on a variety of platforms. For example, an example CNN 1100 may be implemented using a PyTorch library, Adam optimizer and a learning rate of 0.01. In another example, an example CNN 1100 may be implemented using a TensorFlow library. Any other library, optimizer, learning rate, etc. may be used. A loss of function of intersection over union (IOU) may be used. Training may be carried out over a predetermined number of epochs. The predetermined number may be, for example, 100.

Given a finite number of existing CT scans for training the CNN 1100, a predefined percentage of existing CT scans may be used as a training dataset and the remaining may be used for validation. For example, if 450 CT scans are used for training the CNN 1100, 90% (or 405) of the CT scans may be used to train the CNN 1100 and 10% (or 45) may be used to validate the trained CNN 1100.

### EXAMPLE CLAUSES

Example Clause 1: A method may include: receiving a plurality of first three dimensional (3D) models, where each of the first 3D models may include a threshold amount of an area of interest for an anatomical structure; generating, based at least on a portion of the plurality of first 3D models, a plurality of first ultrasound data, where each of the first ultrasound data may include a first instrument position and a first instrument orientation; training, based at least on the plurality of first ultrasound data, an imaging model; receiving a second ultrasound data captured via an imaging instrument having a second instrument position and a second instrument orientation; determining, based at least on the second ultrasound data and the imaging model, that the second ultrasound data does not satisfy a threshold requirement to generate a second 3D model may include the threshold amount; and causing output, based at least on the second ultrasound data and the imaging model, of information indicative of one or more of a change in the second instrument position or the second instrument orientation configured to result in a capture of a third ultrasound data may include the threshold amount.
Example Clause 2: The method of Example Clause 1, where the threshold amount may include 100%.
Example Clause 3: The method of Example Clause 1 or Example Clause 2, where the receiving a second ultrasound data captured via an imaging instrument may include receiving the second ultrasound data in or near real-time as the second ultrasound data is captured via the imaging instrument.
Example Clause 4: The method of any one of Example Clauses 1-3, where the imaging instrument is configured to emit ultrasound waves.
Example Clause 5: The method of any one of Example Clauses 1-4, where the determining that the second ultrasound data does not satisfy a threshold requirement may include determining that the second ultrasound data does not have sufficient planar information for the imaging model to generate a full 3D model.
Example Clause 6: The method of any one of Example Clauses 1-5, where the area of interest for the anatomical structure is a left atrium of a heart.
Example Clause 7: A method may include: receiving a plurality of first three dimensional (3D) models, where each of the first 3D models may include a threshold amount of an area of interest for an anatomical structure; generating, based at least on a portion of the plurality of first 3D models, a plurality of first ultrasound data, where each of the first ultrasound data may include a first instrument position and a first instrument orientation; training, based at least on the plurality of first ultrasound data, an imaging model; receiving a second ultrasound data captured via an imaging instrument having a second instrument position and a second instrument orientation; determining, based at least on the second ultrasound data and the imaging model, that the second ultrasound data satisfies a threshold requirement to generate a second 3D model may include the threshold amount; and generating, based on the second ultrasound data, a second 3D model may include the threshold amount.
Example Clause 8: The method of Example Clause 7, where the threshold amount may include 100%.
Example Clause 9: The method of Example Clause 7 or Example Clause 8, where the receiving a second ultrasound data captured via an imaging instrument may include receiving the second ultrasound data in or near real-time as the second ultrasound data is captured via the imaging instrument.
Example Clause 10: The method of any one of Example Clauses 7-9, where the imaging instrument is configured to emit ultrasound waves.
Example Clause 11: The method of any one of Example Clauses 7-10, where the determining that the second ultrasound data satisfies a threshold requirement may include determining that the second ultrasound data has sufficient planar information for the imaging model to generate a full 3D model.
Example Clause 12: The method of any one of Example Clauses 7-11, where the area of interest for the anatomical structure is a left atrium of a heart.
Example Clause 13: A method may include: receiving a ultrasound data captured via an imaging instrument having an instrument position and an instrument orientation; determining, based at least on the ultrasound data and an imaging model, that the ultrasound data is not sufficient to generate a three dimensional (3D) model may include a threshold amount of an area of interest for an anatomical structure; and causing output, based at least on the ultrasound data and the imaging model, of information indicative of one or more of a change in the instrument position or the instrument orientation configured to result in a capture of another ultrasound data sufficient to generate a 3D model may include the threshold amount.
Example Clause 14: The method of Example Clause 13, where the imaging instrument is configured to emit ultrasound waves.
Example Clause 15: The method of Example Clause 13 or Example Clause 14, where the imaging model is trained using a plurality of first ultrasound data.
Example Clause 16: The method of any one of Example Clauses 13-15, where the plurality of first ultrasound data may include an associated instrument position and an associated instrument orientation.
Example Clause 17: The method of any one of Example Clauses 13-16, where the plurality of first ultrasound data were generated based on a plurality of 3D models.
Example Clause 18: The method of any one of Example Clauses 13-17, where the plurality of 3D models may include an associated threshold amount of an associated area of interest for an associated anatomical structure.
Example Clause 19: The method of any one of Example Clauses 13-18, where the threshold amount is 100%.
Example Clause 20: The method of any one of Example Clauses 13-19, further may include: generating a new 3D model may include the threshold amount of the area of interest for the anatomical structure; producing at least one new ultrasound data based on the new 3D model; and determining, based on the at least one new ultrasound data, if the determining that the ultrasound data is not sufficient to generate a 3D model may include a threshold amount of the area of interest for the anatomical structure was correct.
Example Clause 21: The method of any one of Example Clauses 13-20, further may include: determining, based on the at least one new ultrasound data, that the determining that the ultrasound data is not sufficient to generate a 3D model may include a threshold amount of the area of interest for the anatomical structure was incorrect.
Example Clause 22: The method of any one of Example Clauses 13-21, further may include: updating the imaging model based on the new 3D model and the at least one new ultrasound data.
Example Clause 23: The method of any one of Example Clauses 13-22, where the area of interest for the anatomical structure is a left atrium of a heart.
Example Clause 24: A system may include: one or more processors configured to: receive a plurality of first three dimensional (3D) models, where each of the first 3D models may include a threshold amount of an area of interest for an anatomical structure; generate, based at least on a portion of the plurality of first 3D models, a plurality of first ultrasound data, where each of the first ultrasound data may include a first instrument position and a first instrument orientation; train, based at least on the plurality of first ultrasound data, an imaging model; receive a second ultrasound data captured via an imaging instrument having a second instrument position and a second instrument orientation; determine, based at least on the second ultrasound data and the imaging model, that the second ultrasound data does not satisfy a threshold requirement to generate a second 3D model may include the threshold amount; and cause output, based at least on the second ultrasound data and the imaging model, of information indicative of one or more of a change in the second instrument position or the second instrument orientation configured to result in a capture of a third ultrasound data may include the threshold amount.
Example Clause 25: The system of Example Clause 24, where the threshold amount may include 100%.
Example Clause 26: The system of Example Clause 24 or Example Clause 25, where the receiving a second ultrasound data captured via an imaging instrument may include receiving the second ultrasound data in or near real-time as the second ultrasound data is captured via the imaging instrument.
Example Clause 27: The system of any one of Example Clauses 24-26, where the imaging instrument is configured to emit ultrasound waves.
Example Clause 28: The system of any one of Example Clauses 24-27, where the determining that the second ultrasound data does not satisfy a threshold requirement may include determining that the second ultrasound data does not have sufficient planar information for the imaging model to generate a full 3D model.
Example Clause 29: The system of any one of Example Clauses 24-28, where the area of interest for the anatomical structure is a left atrium of a heart.
Example Clause 30: A system may include: one or more processors configured to: receive a plurality of first three dimensional (3D) models, where each of the first 3D models may include a threshold amount of an area of interest for an anatomical structure; generate, based at least on a portion of the plurality of first 3D models, a plurality of first ultrasound data, where each of the first ultrasound data may include a first instrument position and a first instrument orientation; train, based at least on the plurality of first ultrasound data, an imaging model; receive a second ultrasound data captured via an imaging instrument having a second instrument position and a second instrument orientation; determine, based at least on the second ultrasound data and the imaging model, that the second ultrasound data satisfies a threshold requirement to generate a second 3D model may include the threshold amount; and generate, based on the second ultrasound data, a second 3D model may include the threshold amount.
Example Clause 31: The system of Example Clause 30, where the threshold amount may include 100%.
Example Clause 32: The system of Example Clause 30 or Example Clause 31, where the receiving a second ultrasound data captured via an imaging instrument may include receiving the second ultrasound data in or near real-time as the second ultrasound data is captured via the imaging instrument.
Example Clause 33: The system of any one of Example Clauses 30-32, where the imaging instrument is configured to emit ultrasound waves.
Example Clause 34: The system of any one of Example Clauses 30-33, where the determining that the second ultrasound data satisfies a threshold requirement may include determining that the second ultrasound data has sufficient planar information for the imaging model to generate a full 3D model.
Example Clause 35: The system of any one of Example Clauses 30-34, where the area of interest for the anatomical structure is a left atrium of a heart.
Example Clause 36: A system may include: one or more processors configured to: receive a ultrasound data captured via an imaging instrument having an instrument position and an instrument orientation; determine, based at least on the ultrasound data and an imaging model, that the ultrasound data is not sufficient to generate a three dimensional (3D) model may include a threshold amount of an area of interest for an anatomical structure; and cause output, based at least on the ultrasound data and the imaging model, of information indicative of one or more of a change in the instrument position or the instrument orientation configured to result in a capture of another ultrasound data sufficient to generate a 3D model may include the threshold amount.
Example Clause 37: The system of Example Clause 36, where the imaging instrument is configured to emit ultrasound waves.
Example Clause 38: The system of Example Clause 36 or Example Clause 37, where the imaging model is trained using a plurality of first ultrasound data.
Example Clause 39: The system of any one of Example Clauses 36-38, where the plurality of first ultrasound data may include an associated instrument position and an associated instrument orientation.
Example Clause 40: The system of any one of Example Clauses 36-39, where the plurality of first ultrasound data were generated based on a plurality of 3D models.
Example Clause 41: The system of any one of Example Clauses 36-40, where the plurality of 3D models may include an associated threshold amount of an associated area of interest for an associated anatomical structure.
Example Clause 42: The system of any one of Example Clauses 36-41, where the threshold amount is 100%.
Example Clause 43: The system of any one of Example Clauses 36-42, where the one or more processors are further configured to: generate a new 3D model may include the threshold amount of the area of interest for the anatomical structure; produce at least one new ultrasound data based on the new 3D model; and determine, based on the at least one new ultrasound data, if the determining that the ultrasound data is not sufficient to generate a 3D model may include a threshold amount of the area of interest for the anatomical structure was correct.
Example Clause 44: The system of any one of Example Clauses 36-43, where the one or more processors are further configured to: determine, based on the at least one new ultrasound data, that the determining that the ultrasound data is not sufficient to generate a 3D model may include a threshold amount of the area of interest for the anatomical structure was incorrect.
Example Clause 45: The system of any one of Example Clauses 36-44, where the one or more processors are further configured to: update the imaging model based on the new 3D model and the at least one new ultrasound data.
Example Clause 46: The system of any one of Example Clauses 36-45, where the area of interest for the anatomical structure is a left atrium of a heart.
Example Clause 47: The method of any one of Example Clauses 1-23, where the ultrasound data represents one or more of one or more four dimensional (4D) volumetric images, one or more two dimensional (2D) images, or one or more multi-plane images.
Example Clause 48: system of any one of Example Clauses 24-46, where the ultrasound data represents one or more of one or more four dimensional (4D) volumetric images, one or more two dimensional (2D) images, or one or more multi-plane images.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

1. A method comprising:
receiving a plurality of first three dimensional (3D) models, wherein each of the first 3D models comprises a threshold amount of an area of interest for an anatomical structure;
generating, based at least on a portion of the plurality of first 3D models, a plurality of first ultrasound data, wherein each of the first ultrasound data comprises a first instrument position and a first instrument orientation;
training, based at least on the plurality of first ultrasound data, an imaging model;
receiving second ultrasound data captured via an imaging instrument having a second instrument position and a second instrument orientation;
determining, based at least on the second ultrasound data and the imaging model, that the second ultrasound data does not satisfy a threshold requirement to generate a second 3D model comprising the threshold amount; and
causing output, based at least on the second ultrasound data and the imaging model, of information indicative of one or more of a change in the second instrument position or the second instrument orientation configured to result in a capture of a third ultrasound data comprising the threshold amount.

2. The method of claim 1, wherein the threshold amount comprises 100%.

3. The method of claim 1, wherein the receiving a second ultrasound data captured via an imaging instrument comprises receiving the second ultrasound data in or near real-time as the second ultrasound data is captured via the imaging instrument.

4. The method of claim 1, wherein the imaging instrument is configured to emit ultrasound waves.

5. The method of claim 1, wherein the determining that the second ultrasound data does not satisfy a threshold requirement comprises determining that the second ultrasound data does not have sufficient planar information for the imaging model to generate a full 3D model.

6. The method of claim 1, wherein the area of interest for the anatomical structure is a left atrium of a heart.

7. The method of claim 1, wherein the first ultrasound data represents one or more of one or more four dimensional (4D) volumetric images, one or more two dimensional (2D) images, or one or more multi-plane images.

8. A method comprising:
receiving a plurality of first three dimensional (3D) models, wherein each of the first 3D models comprises a threshold amount of an area of interest for an anatomical structure;
determining, based at least on a portion of the plurality of first 3D models, first ultrasound data, wherein the first ultrasound data comprises a first instrument position and a first instrument orientation;
training, based at least on the first ultrasound data, an imaging model;
receiving a second ultrasound data captured via an imaging instrument having a second instrument position and a second instrument orientation;
determining, based at least on the second ultrasound data and the imaging model, that the second ultrasound data satisfies a threshold requirement to generate a second 3D model comprising the threshold amount; and
generating, based on the second ultrasound data, a second 3D model comprising the threshold amount.

9. The method of claim 8, wherein the threshold amount comprises 100%.

10. The method of claim 8, wherein the receiving a second ultrasound data captured via an imaging instrument comprises receiving the second ultrasound data in or near real-time as the second ultrasound data is captured via the imaging instrument.

11. The method of claim 8, wherein the imaging instrument is configured to emit ultrasound waves.

12. The method of claim 8, wherein the determining that the second ultrasound data satisfies a threshold requirement comprises determining that the second ultrasound data has sufficient planar information for the imaging model to generate a full 3D model.

13. The method of claim 8, wherein the area of interest for the anatomical structure is a left atrium of a heart.

14. The method of claim 8, wherein the first ultrasound data represents one or more of one or more four dimensional (4D) volumetric images, one or more two dimensional (2D) images, or one or more multi-plane images.

15. A method comprising:
receiving ultrasound data captured via an imaging instrument having an instrument position and an instrument orientation;
determining, based at least on the ultrasound data and an imaging model, that the ultrasound data is not sufficient to generate a three dimensional (3D) model comprising a threshold amount of an area of interest for an anatomical structure; and
causing output, based at least on the ultrasound data and the imaging model, of information indicative of one or more of a change in the instrument position or the instrument orientation configured to result in a capture of another ultrasound data sufficient to generate a 3D model comprising the threshold amount.

16. The method of claim 15, wherein the imaging instrument is configured to emit ultrasound waves.

17. The method of claim 15, wherein the imaging model is trained using a plurality of first ultrasound data.

18. The method of claim 17, wherein the plurality of first ultrasound data comprises an associated instrument position and an associated instrument orientation.

19. The method of claim 18, wherein the plurality of first ultrasound data were generated based on a plurality of 3D models.

20. The method of claim 19, wherein the plurality of 3D models comprises an associated threshold amount of an associated area of interest for an associated anatomical structure.

21. The method of claim 20, wherein the threshold amount is 100%.

22. The method of claim 15, further comprising:
generating a new 3D model comprising the threshold amount of the area of interest for the anatomical structure;
producing at least one new ultrasound data based on the new 3D model; and
determining, based on the at least one new ultrasound data, if the determining that the ultrasound data is not sufficient to generate a 3D model comprising a threshold amount of the area of interest for the anatomical structure was correct.

23. The method of claim 22, further comprising: determining, based on the at least one new ultrasound data, that the determining that the ultrasound data is not sufficient to generate a 3D model comprising a threshold amount of the area of interest for the anatomical structure was incorrect.

24. The method of claim 23, further comprising: updating the imaging model based on the new 3D model and the at least one new ultrasound data.

25. The method of claim 15, wherein the area of interest for the anatomical structure is a left atrium of a heart.

26. The method of claim 15, wherein the ultrasound data represents one or more of one or more four dimensional (4D) volumetric images, one or more two dimensional (2D) images, or one or more multi-plane images.
